(19) United States
(12) Patent Application Publication
(10) Pub. No.: US 2007/0254861 A1
(43) Pub. Date: Nov. 1, 2007

(54) Methods for the prevention and/or treatment of peripheral arterial disease

(75) Inventors: Tomihisa Kawasaki, Chuo-ku (JP); Hajime Takamatsu, Chuo-ku (JP); Toshio Uemura, Chuo-ku (JP)

Correspondence Address: SUGHRUE-265550, 2100 PENNSYLVANIA AVE. NW, WASHINGTON, DC, 20037-3213, US

(73) Assignee: Astellas Pharma Inc., Chuo-ku (JP)

(21) Appl. No.: 11/712,397

(22) Filed: Mar. 1, 2007

(30) Foreign Application Priority Data
Apr. 28, 2006 (JP) 2006-124797
Aug. 4, 2006 (JP) 2006-213356

(51) Int. Cl. A61K 31/395; A61P 9/08

(52) U.S. Cl. 514/183

(57) ABSTRACT

A method for the prevention and/or treatment of peripheral arterial disease by compound (I) or its pharmaceutically salts are provided. The compound (I) or its pharmaceutically salts have inhibitory activity against heterotrimeric G protein Gq/11.

### TECHNICAL FIELD

This invention contributes to medical treatments, especially, to the prevention and/or treatment of pain at rest or ulcer/necrosis for patients with PAD.

### BACKGROUND ART

Peripheral arterial disease (PAD) is caused by atherosclerotic lesions of main arteries in legs. PAD patients often claim ischemic symptoms in legs (Lancet, 358, 1257, 2001).

The degree of severity of PAD is most often classified using the system as developed by Fontain, which rates the PAD in four stages based on signs and symptoms: asymptomatic patients (stage I), intermittent claudication (stage II), rest pain (stage III), and tropic lesions (stage IV). PAD is most common in smoking male patients older than 50 years. Patients with PAD as well as with other arterial diseases are progressively increasing by changing food and automotive lives.

PAD is often accompanied by hypertension, diabetes mellitus or hyperlipidemia. Since PAD is derived from systemic atherosclerosis as described above, PAD patients often suffer occlusive vessel diseases in several organs such as ischemic heart diseases, cerebral vessel diseases or renal function disorders. More recently insulin resistance has been thought to play an important role in pathophysiology of PAD as a risk factor. In Japan, especially, patients with diabetes mellitus are increasing year by year as PAD patients are. It is noted that critical limb ischemia (CLI) are increasing as dialysis rates in diabetes kidney diseases are, and that more than 35% of PAD patients are suffered from diabetes mellitus.

Concerning the diagnosis and treatment for PAD patients, Trans Atlantic Inter-Society Consensus (TASC) defined a guideline (Journal of Vascular Surgery, 31, S9, 2000). Severe PAD patients, who have pain at rest or ulcer/necrosis, occupy about 25% of all PAD patients. Though in CLI patients adequate treatments are being selected by judging the degree of ischemic severity, position and area of ischemia, the severity of complications, quality of life and so on, medical treatments to open the occluded vessels by medicines and physical methods are being performed.

In Japan, prostaglandin E1 (PGE1) or argatroban are used for the treatment of patients with severe PAD. PGE1 shows potent vasodilative effects, but has weak anti-platelet aggregation effects. In contrast, argatroban strongly inhibits not only thrombin-dependent platelet activation/aggregation but also blood coagulation, but does not have vasodilative effects.

On the other hand, as physical methodologies for reperfusion of occluded vessels, bypass surgery and percutaneous transluminsal angioplasty (PTA) using such as balloon, laser, atherectomy, stent and so on have been performed. However, PTA or stent placement induces mechanical injuries to vessel tissues including endothelial cells, and thrombotic occlusion in acute phase or restenosis in chronic phase occur. Since platelets are thought to play crucial role in these thrombotic side-effects after angioplasty, platelet aggregation inhibitors such as clopidogrel(=(+)-(S)-methyl 2-(2-chlorophenyl)-2-(6,7-dihydrothieno[3,2-c]pyridin-5(4H)-yl)acetate) and ticlopidine are used (New England Journal of Medicine, 344, 1608, 2001). Therefore, drugs which have both potent anti-platelet aggregation and vasodilative effects are thought to be useful for the treatment of PAD.

The compound (I) which are designated as YM-254890 are products produced by microorganisms of the genus Chromobacterium sp. QS3666 [deposited with National Institute of Bioscience and Human Technology Agency of Industrial Science and Technology (formerly Fermentation Research Institute Agency of Industrial Science and Technology), at 1-3, Higashi 1-chome, Tsukuba-shi, Ibaraki, Japan, accession number FERM BP-10786]. The Compound (I), which inhibits ADP-induced platelet aggregation in human, was isolated from the culture broth of Chromobacterium sp. strain QS3666 in Okutama-Cho of Tokyo (JP-2003-210190; Journal of Antibiotics, 56, 358, 2003 et al.). The structure of this compound (I) was determined using Marfey's method and chiral HPLC analysis (Tetrahedron, 59, 4533-4538, 2003).

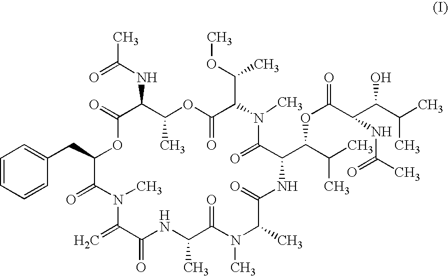

Compound (I) is a specific Gq/11 inhibitor (Journal of Biological Chemistry, 279, 47438, 2004). Gq/11 is well known to be a kind of G proteins. G proteins are heterotrimeric proteins which consist of α, β And γ subunits, and are classified into 4 sub-families of Gs, Gi, Gq and G12 by the homologies of amino acid sequences and its effector protein. Furthermore, each subfamily has several subtypes. The Gq subfamily consists of several subtypes such as Gq, G11, G14, G15 and G16. Though cholera toxin and pertussis toxin are known as substances which modulate and inhibit the function of G protein, no low molecular inhibitor has been reported so far. Compound (I) is a specific Gq/11 inhibitor which strongly inhibits both Gq and G11, but not G14, G15 and G16. Compound (I) inhibits platelet aggregation and platelet-rich thrombus formation on collagen-coated surface under high-shear stress, and exerts potent antithrombotic and thrombolytic effects in in vivo animal thrombosis models (Thrombosis and Haemostasis, 94, 184, 2005, Thrombosis and Haemostasis, 90, 406, 2003). Furthermore, compound (I) inhibits neointima formation 3 weeks after vascular injury in mice (Thrombosis and Haemostasis, 94, 184, 2005). However, the usefulness or effectiveness of compound (I) for patients with PAD has not reported so far.

### DISCLOSURE OF THE INVENTION

The aim of this invention is to offer a drug which has potent platelet aggregation inhibitory and vasodilating effects, to provide an agent which has pharmacological and safety profiles useful for patients with severe PAD, and to suggest a useful usage of a Gq/11 inhibitor.

The inventors found that the local administration of a specific Gq/11 inhibitor was useful for patients with PAD such as arteriosclerosis obliterans (ASO) or Buerger Disease.

The inventors found that compound (I) or its salt was effective for vasoconstriction induced by various vasoconstrictor.

The compound (I) concentration-dependently inhibited vasoconstrictions induced by direct α1 agonist phenylephrine, serotonin (5-HT) and endothelin (ET-1) released from endothelial cells, but not by potassium chloride (KCl). Inhibitory concentration (IC₅₀) values for vasoconstrictions induced by phenylephrine, 5-HT and ET-1 were 8.6 to 18 nM. Compound (I) caused concentration-dependent relaxation on isolated rat aorta without endothelium that had been pre-contracted with 1 μM phenylephrine, with an IC₅₀ value of 11 nM, which is similar with an IC₅₀ value obtained with endothelium, suggesting that compound (I) has a direct vasodilative effect on vascular smooth muscle cells. High concentrations of plasma 5-HT have been observed in patients with peripheral arterial disease (European Journal of Clinical Investigation, 18, 399, 1988). In addition, platelet responses to 5-HT are higher in the elderly and patients with cardiovascular disease than in young, healthy people (Drugs, 36 (suppl. 1), 87, 1988). Furthermore, the presence of ET-1 and its receptors has been reported in segments of femoral artery obtained from patients undergoing procedures for peripheral arterial disease (Journal of Cardiovascular Pharmacology, 36 (5 suppl. 1), S93, 2000). These results suggest that compound (I) is a useful vasodilator for patients with PAD.

Furthermore, these inventors found that compound (I) or its salt is an effective inhibitor of platelet aggregation after injection into the femoral artery.

The inhibitory activity of compound (I) on platelet aggregation after i.a. bolus injection in femoral artery was almost the same as that obtained in a previous i.v. study (Thrombosis and Haemostasis, 90, 406, 2003). Its inhibitory effect is based on the inhibition of the Gq-coupled P2Y1 receptor on platelets. Pharmacokinetic studies using rats demonstrated that, although plasma concentration became undetectable in the blood soon after i.v. bolus injection (T_{1/2}=3.7 minutes), ADP-induced platelet aggregation remained, suggesting that its inhibitory effect on platelet aggregation is based on the inhibition of Gq in platelets.

These inventors found that compound (I) or its salt dose-dependently inhibited the progress of the lesion on lauric acid-induced PAD in rats.

In this study, a lauric acid-induced peripheral arterial injury model in rats was used as a severe PAD model. The direct injection of lauric acid into the artery causes injury of the endothelium, and the subsequent platelet aggregation and platelet adhesion greatly hindered peripheral circulation. This model has been widely applied as a useful tool for the evaluation of antithrombotic agents, and the preventive effects of several agents such as ticlopidine (Thrombosis Research 18, 55, 1980), PGE₁ (Prostaglandins 49, 175, 1995), and 5-HT receptor antagonists (Journal of Cardiovascular Pharmacology 35, 323, 2000) have been studied and reported. Most of these studies investigated the effectiveness of agents administered before the vascular injury, and inhibitory effects of these agents on lesion progression decreased when administered after the lauric acid injection (Journal of Cardiovascular Pharmacology 35, 323, 2000; Arzneimittelforschung 39, 856, 1989). Because the lauric acid causes severe endothelium damage, conventional drugs don't exert sufficient preventive effects in this model. Interestingly, compound (I) dose-dependently inhibited lesion progress, even when administered 15 minutes after the lauric acid injection. Of course compound (I) potently inhibited lesion progress when administered before the lauric acid injection (European Journal of Pharmacology 536, 154, 2006).

Our results suggest that both platelet aggregation and vasoconstriction are major contributing factors for initiation and progression in this model. Since compound (I) is a potent vasorelaxant and anti-platelet aggregation agent, it may prove to be effective as a treatment for PAD in humans.

The inventors found that compound (I) or its salt improved the decreased dermal blood flow induced by lauric acid injection. In contrast, PGE₁ caused a marked increase in dermal blood flow after i.v. infusion, but it failed to improve blood flow after the lauric acid injection. Though direct vasorelaxant effects have not been reported for clopidogrel, a slight improvement in the lauric acid-affected blood flow was observed in this study, probably due to clopidogrel's potent anti-platelet aggregation effect. In the Example 4, PGE₁ and clopidogrel (only when administered before the lauric acid injection) significantly prevented lesion progression 3rd days after the lauric acid injection. PGE1 has vasodilative effects, but its anti-aggregating effects of platelets are weak. Clopidogrel potently inhibits platelet aggregation, but doesn't have vasodilative effects. Compound (I) exerted potent efficacies in this model, in which conventional drugs don't produce significant effects, based on the fact that compound (I) has both a very potent vasodilative and anti-platelet aggregation effects.

These inventors found that compound (I) or its salt dose-dependently decreased the systemic mean blood pressure after the injection into a femoral artery of rats in an increasing fashion. The significant hypotensive doses after i.a. bolus injection of compound (I) in this study were almost the same as those obtained after i.v. bolus injection in the previous study (Thrombosis and Haemostasis 90, 406, 2003).

This hypotensive effect may be due to the inhibition of Gq/11-coupled vasculature signaling. It was noted that the significant hypotensive dose (30 μg/kg i.a.) of compound (I) in the Example 6 was 10 times higher than the dose which produced a significant preventive effect in the Example 4.

Though the safety margin between preventive doses in this model and hypotensive doses when administered either orally or intravenously was 1 to 3 times (Thrombosis and Haemostasis 90, 406, 2003; Thrombosis and Haemostasis 94, 184, 2005), the intra-arterial (i.a.) local administration of compound (I) produced a more effective preventive properties with wider safety margin.

### BRIEF DESCRIPTION OF THE DRAWINGS

By way of example and to make the description more clear, reference is made to the accompanying drawing in which:

FIGS. 1A and 1B show the vasorelaxant effect of compound (I) on vasoconstruction in isolated rat aorta induced various vasoconstruction (Example 2).

FIG. 2 shows the effect of compound (I) on ex vivo platelet aggregation inhibition after single administration in the rat femoral artery (Example 3).

FIG. 3 shows the inhibitory effect of compound (I) on lesion progression in a rat peripheral arterial disease model induced by lauric acid (Example 4).

FIG. 4 shows the effect of compound (I) on blood pressure and heart rate after single bolus injection in femoral artery of rats (Example 6).

### BEST MODE FOR CARRYING OUT OF THE INVENTION

As hereunder, the present invention will be specifically illustrated by way of Examples although the present invention is not limited by those Examples at all.

In some cases, the compound of the present invention forms a salt and, so far as such a salt is pharmaceutically acceptable, that is included in the present invention. Its specific examples are salt with inorganic acid such as hydrochloric acid, hydrobromic acid, hydroiodic acid, sulfuric acid, nitric acid and phosphoric acid, etc; an acid-addition salt with organic acid such as formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, aspartic acid and glutamic acid, etc. The present invention further includes various kinds of hydrate and solvate of the compound of the present invention and a pharmaceutically acceptable salt and a substance having crystal polymorphism as well.

Depending upon the type of the substituent, the compound of the present invention represented by the formula (I) may contain asymmetric carbon atom and there may be optical isomer due to that. The present invention includes all of those optical isomers both as a mixture thereof and as isolated ones. In some cases, tautomers may be present in the compound of the present invention and the present invention includes all of those tautomers both as isolated ones and as a mixture thereof.

Injection for parenteral administration includes aseptic aqueous or non-aqueous solution, it includes suspension and emulsion. Aqueous solution and suspension may contain distilled water for injection and physiological saline for example. Examples of non-aqueous solution and suspension are propylene glycol, polyethylene glycol, plant oil such as olive oil, alcohol such as EtOH and Polysolvate 80. Such a composition may further contain adjuvant such as antiseptic, moisturizer, emulsifier, dispersing agent, stabilizer and dissolving aid. They are sterilized by, for example, filtration through a bacteria-retaining filter or compounding or irradiation of bactericide. They may be also used in such a manner that an aseptic solid composition is manufactured and, before use, it is dissolved in an aseptic water or in an aseptic solvent for injection.

The dose is appropriately decided depending upon each case by taking route of administration, symptom, age, sex, etc. into consideration. In the case of a common intra-arterial (i.a.) administration, it is appropriate that the daily dose is about 0.01 to 100 mg/body weight, preferably about 0.05 to 5 mg/body weight and it is administered once or divided into two to four times in a day to administer.

### EXAMPLE 1

Drug Preparation for Intra-Arterial Injection

### Material(s) and Method(s)

The compound (I) was dissolved in 100% ethanol and adjusted to 20 mg/ml. Appropriate concentrations of compound (I) were prepared using saline at final concentrations of 0.5% ethanol.

### EXAMPLE 2

Vasorelaxant Effect of Compound (I) on Vasoconstruction in Isolated Rat Aorta Induced Various Vasoconstruction

### Material(s) and Method(s)

Four male SD rats (Japan CLEA) were used in each group of this experiment. Rats were anesthetized with pentobarbital (60 mg/kg i.p.) and euthanized via exsanguination. The aortas were isolated and all adjacent tissue was removed. Rings of aorta, approximately 2-3 minutes length, were suspended in 10-ml organ baths containing Krebs-Henseleit solution (henceforth referred to as Krebs) of the following composition (mM): NaCl, 112.0; KCl, 4.7; KH₂PO₄, 1.2; MgSO₄, 1.2; CaCl₂, 2.5; NaHCO₃, 25.0; glucose, 11.0. The Krebs was maintained at 37±1° C. and aerated with a gas mixture of 95% O₂:5% CO₂ (pH 7.4). Experiments were conducted on aortic tissue of two types. For type 1, the endothelium was removed by gently rubbing the internal surface of the vessel. For type 2, care was taken to maintain the integrity of the endothelium. One ring was placed on a hook that was suspended from a force displacement transducer (SB-1T, Nihon Kohden, Tokyo, Japan), 1.0 g tension was applied, and changes in the force of contraction were isometrically measured. Following a 1-h equilibration period, the rings were pretreated with 1 μM phenylephrine. Compound (I) was dissolved in dimethyl sulfoxide (DMSO) and added to the baths (0.1% DMSO, v/v), after which a cumulative compound (I) (1-100 nM) concentration-response curve was constructed for each type of ring. Contractions were caused in aorta rings with endothelium using three different compounds: 10 μM serotonin (5-HT), 30 nM endothelin-1 (ET-1), and 60 mM KCl. The vasorelaxant effect of compound (I) on the contractions caused by each of these compounds was then compared. Various concentrations of compound (I) were added 30 minutes before treatment with each vasoconstrictor. For 5-HT and KCl, the concentration-inhibition rate was measured for each tissue. For ET-1, each tissue was used to measure the inhibition rate in response to a given concentration of compound (I).

### Result(s)

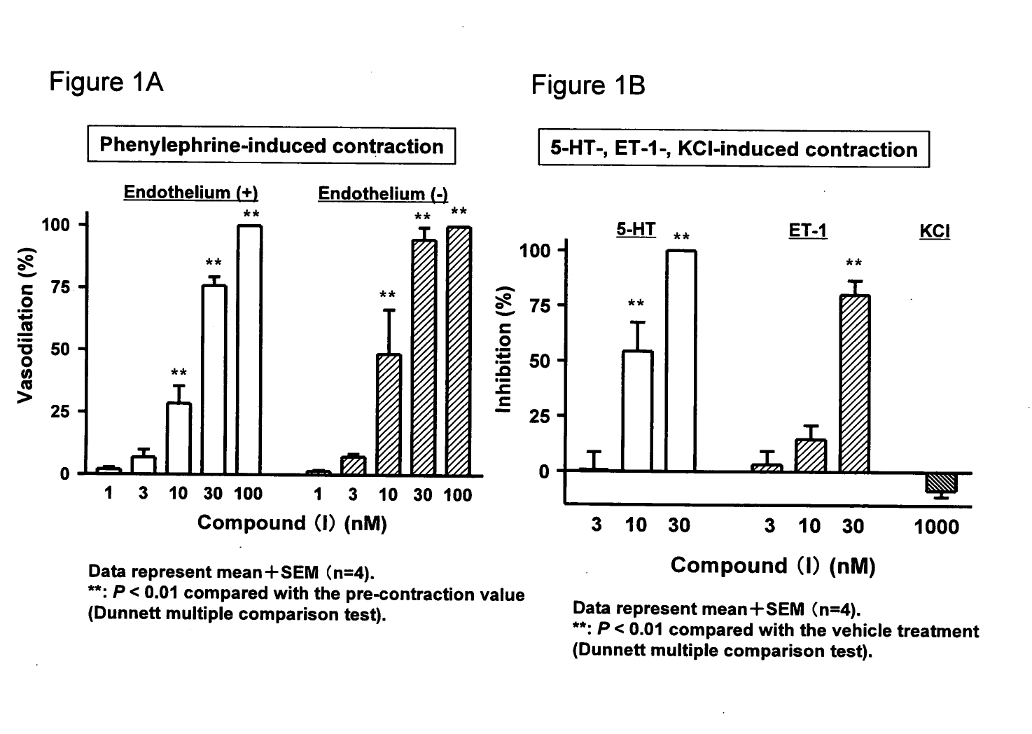

As shown in FIGS. 1A and 1B, compound (I) caused concentration-dependent relaxation on isolated rat aorta both with and without endothelium that had been pre-contracted with 1 μM phenylephrine, with IC₅₀ values of 16±2.3 nM (n=4, mean±SEM) and 11±2.9 nM (n=4, mean±SEM), respectively. Furthermore, compound (I) concentration-dependently inhibited contractions induced by 5-HT and ET-1 in isolated rat aorta. Compound (I) inhibited vascular contraction induced by 10 μM 5-HT and 30 nM ET-1 in a concentration-dependent manner with IC₅₀ values of 8.6±1.0 nM (n=4, mean±SEM) and 18 nM (n=4, mean), respectively. In contrast, compound (I) was ineffective against the 60 mM KCl-induced contractions, even at a concentration of 1 μM.

### EXAMPLE 3

Effect of Compound (I) on Ex Vivo Platelet Aggregation Inhibition After Single Administration in the Rat Femoral Artery

### Material(s) and Method(s)

Four male SD rats (Japan CLEA) were used in each group of this experiment. All animals were fasted overnight before the experiment. After being anesthetized with pentobarbital (60 mg/kg i.p.), vehicle (5% ethanol) or compound (I) was injected into either the left femoral artery or the right jugular vein via a catheter. The rats were anesthetized with pentobarbital (60 mg/kg i.p.) before blood sampling. Blood was collected from the vena cava in syringes containing 3.8% sodium citrate at the following time points: 5 minutes after an intra-arterial (i.a.) single bolus injection of compound (I) (1-10 μg/kg). Platelet-rich plasma was obtained by centrifuging the blood at 200×g for 5 minutes at ambient temperature. This residue was further centrifuged at 2,000×g for 10 minutes in order to obtain platelet-poor plasma. Platelet counts were measured with an automatic cell counter (MEK-6258, Nihon Kohden, Tokyo, Japan), and adjusted to 3×10⁵/μl with platelet-poor plasma. Platelet aggregation in platelet-rich plasma was measured using an aggregometer (MCM Hema Tracer 212, MC Medical, Tokyo, Japan) to record the increase in light transmission detected through a stirred suspension maintained at 37° C. for 5 minutes. Platelet aggregation was induced in 90 μl of platelet-rich plasma (3×10⁵/μl) by adding 10 μl of 50 μM ADP. The inhibitory rate was calculated by dividing the absorbance area for the mixture containing the test sample by the absorbance area obtained for the vehicle-treated group.

### Result(s)

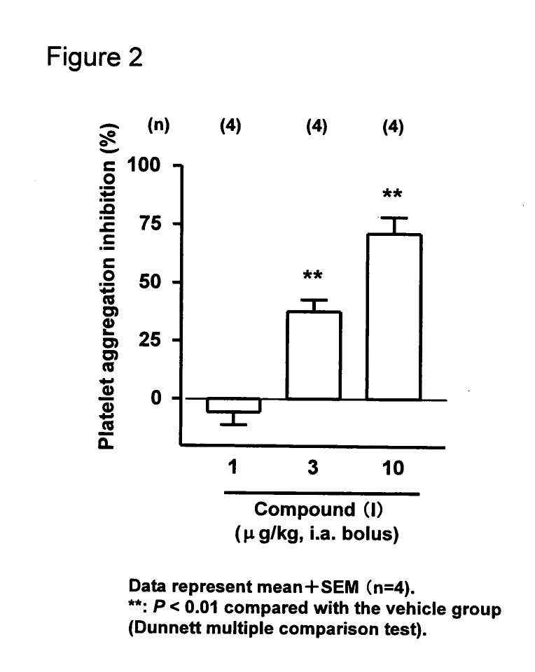

As shown in FIG. 2, compound (I) dose-dependently inhibited ADP-induced platelet aggregation 5 minutes after an i.a. bolus injection. Its inhibitory rates at 1, 3 and 10 μg/kg were −5.6±5.4%, 38±5.1%, and 71±7.0%, respectively (n=4, mean±SEM).

### EXAMPLE 4

Inhibitory Effect of Compound (I) on Lesion Progression in a Rat Peripheral Arterial Disease Model Induced by Lauric Acid

### Material(s) and Method(s)

A rat peripheral arterial disease model was produced by using a lauric acid injection method modified from that of Ashida et al. (Thrombosis research, 18, 55, 1980). Six to 24 animals were used in each group of this experiment. After being fasted overnight, each rat was anesthetized with pentobarbital (60 mg/kg i.p.), and the left femoral artery was freed from the surrounding tissue. The femoral artery was cannulated with a polyethylene catheter for i.a. injection of the lauric acid and the test drug. For the i.v. infusion of PGE₁, the jugular vein was cannulated with a polyethylene catheter. To induce peripheral arterial vascular injury, 0.33 ml/kg of lauric acid (7.5 mg/ml in distilled water) was injected into the distal side of the arterial. Compound (I) was administered in a single i.a. bolus injection 15 minutes after the lauric acid injection. PGE₁ was administered either intravenously for 30 minutes at 1 μg/kg/minutes starting 5 minutes before the lauric acid injection or intra-arterially for 15 minutes at 0.2 μg/kg/minutes starting 5 minutes after the lauric acid injection. Clopidogrel was administered orally either at 3 and 30 mg/kg 4 hours before the lauric acid injection, or at 30 mg/kg 2 hours after the lauric acid injection. It was also administered orally once a day for 3 days after the lauric acid injection. The treated hindlimb was examined macroscopically on days 3 after the lauric acid injection. The progress of the lesion was assessed using the following 5-point graded scoring system: grade 0: normal appearance, grade 1: the affected region was limited to the nails, grade 2: the affected region was limited to the fingers, grade 3: either the fingers are beginning to fall off or lesions appear on the paw, grade 4: either the paw is beginning to fall off or lesions appear on the leg. The condition of each toe was assessed and scored, and the sum of the scores for the five toes was used as the lesion index. If a lesion developed on the sole of the foot, 5 more points were added.

### Result(s)

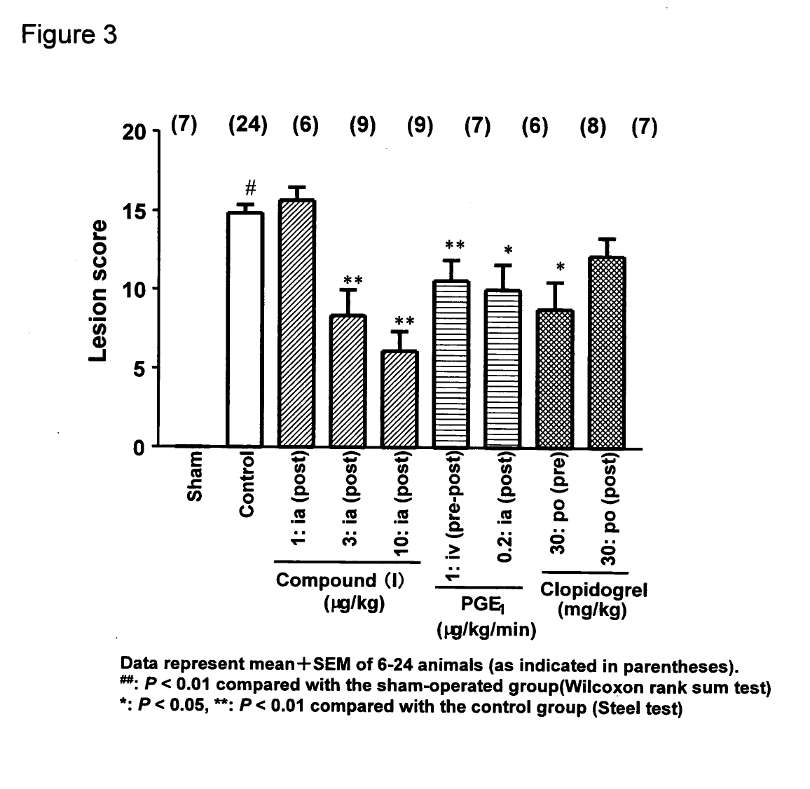

As shown in FIG. 3, no lesion progression was observed in the sham operated animals. In contrast, three days after the lauric acid injection, the paw became gangrenous and then mummified in the control group. Compound (I) inhibited lesion progression, with significance, at 3 μg/kg or more when administered 15 minutes after the lauric acid injection. PGE₁ significantly inhibited lesion progression after i.v. infusion at 1 μg/kg/minutes or i.a. infusion at 0.2 μg/kg/minutes. The efficacy of clopidogrel was detected when it was administered 4 hours before the lauric acid injection, but no effect was observed when administered after the lauric acid injection.

### EXAMPLE 5

Effect of Compound (I) on Decreased Blood Flow in the Rat Femoral Artery after the Lauric Acid Injection

### Material(s) and Method(s)

Three male SD rats (Japan CLEA) were used in each group of this experiment. A laser Doppler blood flow meter (Laser Doppler Perfusion Imager System, Lisca) was used to evaluate the perfusion in the left (ischemic) and right (non-ischemic) rat hindlimbs. Before and during scanning, animals were placed on a heating plate set at 37° C. to minimize variations in temperature. The femoral artery was cannulated with a polyethylene catheter for i.a. injection of the lauric acid and test drug. For i.v. PGE₁ infusion, the jugular vein was cannulated with a polyethylene catheter. Either compound (I) (10 μg/kg) or vehicle was administered in an i.a. bolus injection 15 minutes after the lauric acid injection (0.33 ml/kg, 7.5 mg/ml in distilled water). The laser Doppler images were recorded just before and 10 minutes after the lauric acid injection, as well as 10 minutes after the compound (I) (or vehicle) injection. PGE₁ was administered by i.v. infusion at a rate of 1 μg/kg/minutes for 30 minutes beginning 5 minutes before the lauric acid injection. The laser Doppler images were recorded just before and 5 minutes after the start of infusion (just before the lauric acid injection), and then again 10 minutes after the lauric acid injection. Either vehicle (0.5% methylcellulose solution) or clopidogrel (30 mg/kg) was orally administered 4 hours before the experiment. For the oral studies, the laser Doppler images were recorded just before and 10 minutes after the lauric acid injection.

### Result(s)

Effect of compound (I) on decreased blood flow in the rat femoral artery after the lauric acid injection was judged by perfusion images before and after the lauric acid injection in each treatment group. The perfusion signal was subdivided into 6 separate intervals, with each displayed as a different color. The perfusion values upon which the color-coded pixels are based are stored and remain available for further data analysis. In the vehicle (i.a.) group, dermal blood flow decreased markedly (center panel) immediately after the lauric acid injection, and did not improve at all after vehicle injection. Compound (I) (10 μg/kg), when administered intra-arterially in a bolus 15 minutes after the lauric acid injection, improved the lauric acid-induced reduction in dermal blood flow somewhat, but did not correct it completely. In the PGE₁ group, a marked increase in dermal blood flow was observed in both hindlimbs 5 minutes after the start of the infusion; however, there was no improvement in the blood flow despite continuous infusion of the drug. Oral administration of clopidogrel improved the blood flow only slightly.

### EXAMPLE 6

Effect of Compound (I) on Blood Pressure and Heart Rate after Single Bolus Injection in Femoral Artery of Rats

### Material(s) and Method(s)

Five male SD rats (Japan CLEA) were used in each group of this experiment. The rats, which were fasted overnight, were anesthetized with urethane (1.4 g/kg i.p.) and then secured on an operating table. The left carotid artery was cannulated with polyethylene catheters in order to monitor systemic blood pressure and heart rate. The left femoral artery was likewise cannulated with polyethylene catheters for the injection of compound (I). Systemic blood pressure was measured with a pressure transducer (AP-200T, Nihon Kohden) and heart rate was measured with a tachometer (AT-600G, Nihon Kohden) that was triggered by the arterial pulse wave. Changes in these parameters were continuously monitored by a thermal recorder (WT-685G, Nihon Kohden). Compound (I) was administered in an i.a. bolus injection at the rate of 5 mL/kg. The dosage (10 to 100 μg/kg) was increased every 10 to 20 minutes. The inhibitory rate was calculated by dividing the heart rate and mean blood pressure levels obtained after the compound (I) injection by them obtained after the vehicle injection.

### Result(s)

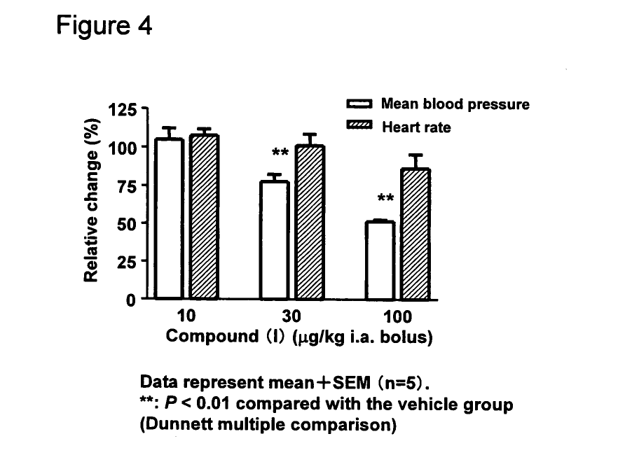

As shown in FIG. 4, compound (I) dose-dependently decreased mean blood pressure. The relative mean blood pressure reductions at doses of 30 and 100 μg/kg i.a. were 25±5.2% and 50±2.1%, respectively. Up to 100 μg/kg, no significant change of the heart rate was observed.

### INDUSTRIAL APPLICABILITY

The compound (I) or its salt exerts potent platelet aggregation inhibition and vasodilative effect. The local administration of this compound (I) can provide safer and more effective treatment for patients with PAD/severe PAD than its systemic administration. This invention produces decrease of pain at rest, prevention of ulcer/necrosis development, decrease of amputation rate. The compound (I) is expected to be effective in the treatment for patients with ASO and Buerger Disease.

While the invention has been described in detail and with reference to specific embodiments thereof, it will be apparent to one skilled in the art that various changes and modifications can be made therein without departing from the scope thereof.

This application is based on Japanese patent applications No. 2006-124797 filed Apr. 28, 2006 and No. 2006-213356 filed Aug. 4, 2006, the entire contents thereof being hereby incorporated by reference.

The patents, patent applications and publications cited herein are incorporated by reference.

1. A method for the prevention and/or treatment of peripheral arterial disease by compound (I) or its pharmaceutically acceptable salt, which is Gq/11 inhibitor.

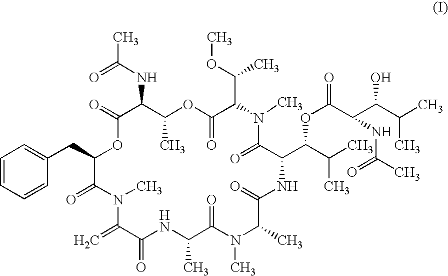

2. A method according claim 1, which is an injectable drug or an eluting agent in drug-eluting stent.

3. A method for the prevention and/or treatment of peripheral arterial disease in a patient, said method comprising administering to said patient a therapeutically effective amount of compound (I) or its pharmaceutically acceptable salt.
